(19) United States
(12) Patent Application Publication
(10) Pub. No.: US 2026/0165913 A1
(43) Pub. Date: Jun. 18, 2026

(54) Apparatus, system and process

(71) Applicant: Hark Medical Research Pty Ltd, Richmond, Victoria (AU)

(72) Inventors: Aron STEG, Melbourne (AU); Matthew George WADE, Melbourne (AU); Tafadzwa SHAAMANO, Melbourne (AU); Darius IGNASIAK, Melbourne (AU); Nicholas David HOWARD, Melbourne (AU); Douglas BUCKLE, Melbourne (AU)

(73) Assignee: Hark Medical Research Pty Ltd, Melbourne (AU)

(21) Appl. No.: 19/125,205

(22) PCT Filed: Oct. 27, 2023
(86) PCT No.: PCT/AU2023/051086

§ 371 Date: Apr. 28, 2025

(30) Foreign Application Priority Data
Oct. 28, 2022 (AU) 2022259848
Oct. 25, 2023 (AU) 2023903408

(51) Int. Cl. A61J 7/00; A61J 1/03; A61J 1/14; A61J 7/04

(52) U.S. Cl. CPC A61J 7/0084; A61J 1/03; A61J 1/1437; A61J 7/0436; A61J 7/0454; A61J 7/0481; A61J 2200/30

(57) ABSTRACT

An apparatus including: a housing for containing/protecting/storing a plurality of containers/packages containing pills/medicaments; a power source in the housing; an actuator arrangement powered by the power source and configured to administer/deliver the pills/medicaments for a pre-selected patient; and a sensor powered by the power source to activate the actuator arrangement to allow the at least one package to be opened/accessed manually when the sensor detects a pre-defined identifier of the pre-selected patient.

### RELATED APPLICATIONS

The present disclosure is related to Australian Provisional Patent Application No. 2022259848, filed 28 Oct. 2022 in the name of Hark Medical Research Pty Ltd, and Australian Provisional Patent Application No. 2023903408, filed 25 Oct. 2023 in the name of Hark Medical Research Pty Ltd, the originally filed patent specifications of which are hereby incorporated by reference in their entireties herein.

### TECHNICAL FIELD

The present disclosure relates to apparatuses, systems and processes for administering/delivering pills/medicaments, particularly for administering/delivering pills/medicaments in containers/packages, including having multiple pills/medicaments per dose, including medical sachet administration/delivery devices.

### BACKGROUND

Medication administering institutions, e.g., hospitals, require a structured system to manage compliance with regulators as well as adherence by their consumers, e.g., 9 out of 10 care institutions reported to rely on analogue reports by consumers or nursing and carer staff for medication management, and there may be only 50% adherence to medication among patients that suffer from chronic diseases. Furthermore, patients are less likely to adhere to their medication where they have greater than 1 pill per dose.

Where patients are required to take more than 1 pill per dose, medication dispensing pharmacies can package pills into blister packs or sachets, for example, to assist patients in adhering to their prescribed medications. The individually packaged doses can be labelled with information such as the date and time at which they should be taken. Despite these measures, patients can still forget to take a dose at the correct time, or may access more doses than are prescribed.

Negative impacts of these problems/shortcomings include increased medication induced mishaps, e.g., hospitalisations, increased dependency on carer staff and/or family, and increased health risks.

It is desired to address or ameliorate one or more of the problems/shortcomings in the prior art, or to at least provide a useful alternative.

### SUMMARY

Described herein is an apparatus (medication administration/delivery device) for administering/delivering medication in containers/packages, each configured to include multiple pills/medicaments per dose (including oral medications), the apparatus including:
a. a (solid/water/tamper-resistant) housing for containing/protecting/storing a plurality of the containers/packages (e.g., 14 or 28 containers, or 28 packages, or a sachet roll of substantially 110-mm diameter) containing the pills/medicaments;
b. a power source (electrical battery and/or supply connection) in the housing;
c. an actuator arrangement (e.g., at least one solenoid and/or at least one (stepper) motor) powered by the power source and configured to administer/deliver the pills/medicaments for a pre-selected patient (e.g., by unlocking/releasing the housing and/or by moving the containers/packages from inside the housing); and
d. at least one sensor powered by the power source to activate the actuator arrangement (e.g., to unlock/release the housing and/or to move the containers/packages from inside the housing) to allow the at least one package to be opened/accessed manually when the sensor detects a pre-defined identifier of the pre-selected patient.

The at least one sensor may include a biometric sensor (optical or touch) and the pre-defined identifier may be a biometric identifier. The at least one sensor may include a radio-frequency identification (RFID) sensor and the pre-defined identifier may be an RFID identifier. The at least one sensor may include a plurality of sensors, including a biometric sensor and a radio-frequency identification (RFID) sensor, for example, and the sensors may activate the actuator arrangement when only one, or only when all, of the sensors detect respective pre-defined identification of the patient.

The apparatus may also include one or more of the following:
a. at least one microcontroller unit (MCU) configured to control the actuator arrangement (to activate the actuator arrangement to unlock or release the housing and/or to move the containers/packages from inside the housing) based on the sensor detecting the pre-defined identifier of the pre-selected patient;
b. the MCU configured to perform a setup process, including receiving a user input from the at least one sensor representing an identifier (input identifier) of the user/patient, and storing the input identifier as the pre-defined identifier;
c. an audio/visual (A/V) system, including an audio speaker and/or a light/display in/on the housing, configured to play/display alerts (local alerts) for the patient;
d. the MCU configured to control the A/V system based on a calendar/clock and a preselected schedule stored in the MCU and/or accessed by the MCU from a remote computing system, which may include cloud servers, wherein the preselected schedule defines reminders to administer/deliver the pills/medicaments based on the preselected schedule (from the patient medication record);
e. at least one network interface module/card (“network interface”) configured to allow electronic/data communication between the MCU and the remote computing system, including via Cellular/Bluetooth/WiFi/Ethernet/Internet protocols-the network interface may include a cellular module configured to connect to a commercial cellular network, e.g., a 4G or a 5G network;
f. the MCU configured to access a patient medication schedule (record of time when to take doses) for the pre-selected patient (stored in the MCU and/or accessed on the remote computing system), wherein the at least one sensor is configured to activate only when sensing the identifier of the patient, wherein the patient medication schedule is provided/sent by a medical provider (pharmacy) that also provides/dispenses the medication in the containers/packages according to the patient medication record; and
g. the MCU configured to send (remote) alerts to the remote computing system for distribution to selected user communication devices (e.g., mobile devices of carers with contact identifiers recorded in the MCU and/or the remote computing system).

Described herein is a system including the apparatus and one or more of the following:
a. a remote computing system with at least one recording/reporting module configured to (i) receive and store usage data from the apparatus representing records of the administering/delivering of the pills/medicaments (including which packages/containers and corresponding dates/times from the calendar/clock), and (ii) generate reports based on the stored records, including reports on adherence,, and analytics (vitals) for access by care institutions, doctors and pharmacists;
b. an application (app) on a handheld smart device (e.g., smart phone) and/or a personal computer (PC) configured to communicate with the remote computing system to receive the remote alerts, and to generate A/V alerts using the display/lights/speaker of the handheld smart device and/or PC to alert a remote user of the handheld smart device/PC (e.g., a carer); and
c. an application on a computing system controlled by a medical provider (e.g., pharmacy, doctor, care institution), wherein the computing system is configured/permitted to access private data of the patient including the patient medication schedule, wherein the application is configured to receive usage data from the apparatus and the reports from the remote computing system, wherein the usage data include records of operation of the at least one sensor to activate the actuator arrangement (identified by patent ID), and/or records of the medication/pills/medicaments being administered/delivered (identified by patent ID), wherein the application is configured to match the records with the patient medication schedule (using the patent ID).

The apparatus may be in the form of a sachet delivery mechanism. The housing dimensions (mm) may be substantially 170L×120W×180H, or substantially 250L×140W×160H.

The apparatus may be in the form of a container (non-sachet) delivery system. The housing dimensions (mm) may be substantially 110L×55W×182H for a 2 by 7 grid of containers, or 130L×55W×207H for a 4 by 7 grid of containers. In other embodiments, the housing dimensions (mm) may be substantially 262L×159W×155H.

Described herein is a process including:
a. containing/protecting/storing a plurality of containers/packages containing pills/medicaments;
b. powering an actuator arrangement to administer/deliver the pills/medicaments for a pre-selected patient (e.g., by unlocking/releasing the housing and/or by moving the containers/packages from inside the housing); and
c. activating the actuator arrangement to allow the at least one package to be opened/accessed manually (e.g., by unlocking/releasing the housing and/or by moving the containers/packages from inside the housing) when a sensor detects a pre-defined identifier of the pre-selected patient.

Described herein is a cartridge for use in a medication administration/delivery apparatus that administers/delivers medication in containers/packages, each configured to include multiple pills/medicaments per dose (including oral medications), the cartridge including:
a. an axle;
b. a lever configured to hold/turn the axle and connected to the axle when in use; and
c. a gripper attached to the axle and configured to grip an edge of a first sachet in a strip of interconnected sachets containing medication/medicament to wind the strip of interconnected sachets onto the axle by holding/turning the lever.

The gripper may deform/perforate the edge of the first sachet. The gripper may include a portion made of a woven material.

The lever may be provided by a disc with a rotational axis aligned with a rotational axis of the axle configured for manual holding/turning of the axle. The disc may include two or more apertures disposed circumferentially around the disc, and these apertures are configured for gripping manually/by a person's finger.

The cartridge may include a cartridge housing, connected to the axle when in use, configured to: contain the strip of interconnected sachets; and allow administration/delivery of the sachets through an aperture in the cartridge housing. The cartridge housing may include one or two apertures arranged to: hold one or both ends of the axle that extend beyond the interconnected sachets when on the axle; and allow rotation of the axle relative to the cartridge housing to unwind the strip of interconnected sachets from the axle.

At least one of the ends of the axle may extend though the cartridge housing to meet the lever such that the lever can hold/turn the axle. As a result, the lever can hold/turn the axle without holding/turning the cartridge housing.

The cartridge may include a handle for lifting/lowering/carrying the cartridge when loaded with the strip of interconnected sachets. The handle may include a hinge connection to the cartridge such that the handle can move between: an open condition for the lifting/lowering/carrying; and a closed condition for storing/resting the cartridge (e.g., on its foot portion on the support surface).

The cartridge may include a foot portion connected to the axle (via the cartridge housing) configured to hold the axle substantially parallel to a support surface when the cartridge is resting on the support surface (by gravity, e.g., on a table/bench and/or in a medication administration/delivery machine). The handle may be connected to an outer surface of the cartridge housing, the handle is disposed substantially opposite the foot portion, and the handle is curved to conform with the outer surface of the cartridge housing when in the closed condition.

The cartridge may include a machine-readable identification device configured to be detected/read by an optical/RF detector/reader for uniquely identifying one of a plurality of the cartridges. The identification device may include an optically readable label (e.g., a barcode or QR code) and/or an RF readable tag (e.g., an RFID tag).

Described herein is a medication administration/delivery apparatus including:
a. a housing for containing/protecting/storing a plurality of interconnected sachets containing pills/medicaments;
b. an actuator arrangement (e.g., a pair of counter-rotating wheels) mounted within the housing configured to administer/deliver the sachets containing pills/medicaments;
c. at least one microcontroller unit (MCU) configured to control the an actuator arrangement (e.g., a motor coupled to one of the wheels); and
d. a sensor arrangement, disposed within the housing, configured to detect an edge of a leading one of the sachets and communicate with the MCU to stop the an actuator arrangement (e.g., the motor) when the sachets are in a starting position defined by a location of the leading edge relative to the an actuator arrangement or the housing.

The sensor arrangement is in the form of a non-contact sensor arrangement that detects the sachets without hindering their movement. The sensor arrangement may include a plurality of non-contact sensors, e.g., at least one of an optical sensor and a motion sensor, e.g., two optical interrupters and one laser motion sensor.

The actuator arrangement may include a pair of counter-rotating wheels and a motor coupled to at least one of the wheels. The counter-rotating wheels in the pair may be biased towards one another in order to grip the interconnected sachets therebetween. One wheel in the pair of wheels may include gripping material (e.g., material with a high coefficient of static friction) to grip the interconnected sachets. One wheel in the pair of wheels may include deformable material (e.g., resilient material) that deforms around the pills/medicaments in the interconnected sachets. The wheels may be configured to distribute pills/medicaments in the interconnected sachets when the pills/medicaments are mutually stacked in the interconnected sachets.

The MCU may be configured to control the actuator arrangement (e.g., the motor0 based on a preselected administration/delivery length stored in or accessed by the MCU.

The apparatus may include an aperture through which the sachets can be administered/delivered by the actuator arrangement. The apparatus may include two pairs of the counter-rotating wheels to direct the interconnected sachets straight towards the aperture.

The apparatus may include a lockout clamp movable between an open position, which opens the aperture, and a closed position, which closes the aperture to block the interconnected sachets moving through the aperture.

Described herein is another process including (e.g., but not necessarily, in the following order):
a. powering the actuator arrangement (e.g., a first actuator) to move at least one of a plurality of interconnected sachets stored within a housing towards an aperture in the housing (thus moving the containers/packages from inside the housing), and/or powering an actuator arrangement (e.g., a first actuator) to move the plurality of interconnected sachets away from the aperture in the housing;
b. sensing, with a plurality of sensors, when the plurality of interconnected sachets is in a pre-defined starting position; and
c. restricting manual access to the interconnected sachets stored within the housing.

The process may also include activating the actuator arrangement (e.g., a second actuator) to unlock or release at least one of the interconnected sachets when a sensor detects a pre-defined identifier of a pre-selected person. The actuator arrangement (e.g., the first actuator) may include at least one wheel.

Described here is yet another process including (e.g., but not necessarily, in the following order):
a. activating an actuator arrangement to unlock or release a housing when a sensor detects a pre-defined identifier of a pre-selected person;
b. powering the actuator arrangement to move at least one of a plurality of sachets stored within the housing towards an position accessible by the person from inside the housing;
c. restricting the person's access to the plurality of sachets except for the at least one sachet;
d. allowing tearing/removing, by the patient, the at least one container/package;
e. powering the actuator arrangement to move the plurality of containers/packages away from the position accessible by the person;
f. sensing, with a plurality of sensors, when the plurality of containers/packages are in a pre-defined starting position; and
g. in response to the sensing of the containers/packages being in the pre-defined starting position, locking the housing to restrict the person's access to the plurality of sachets.

### BRIEF DESCRIPTION OF THE DRAWINGS

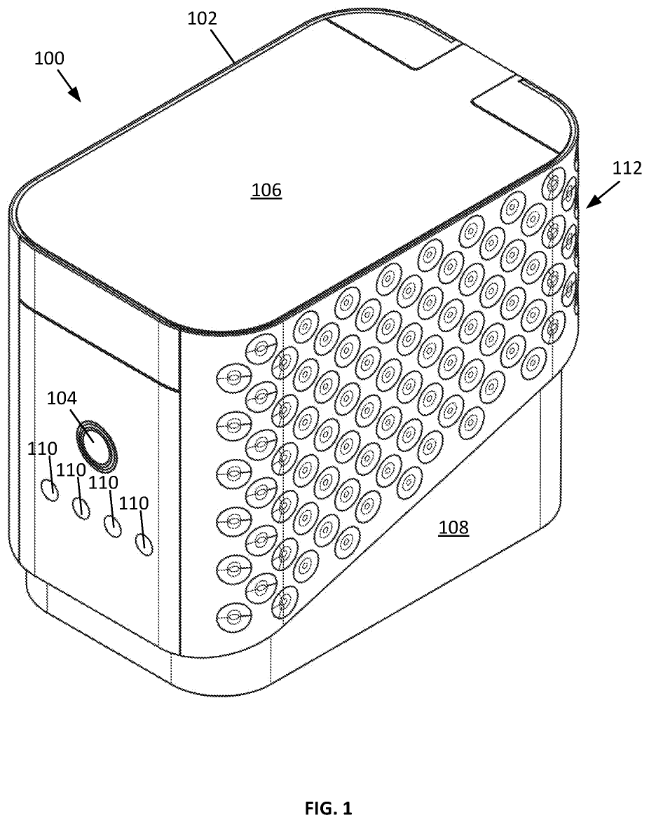

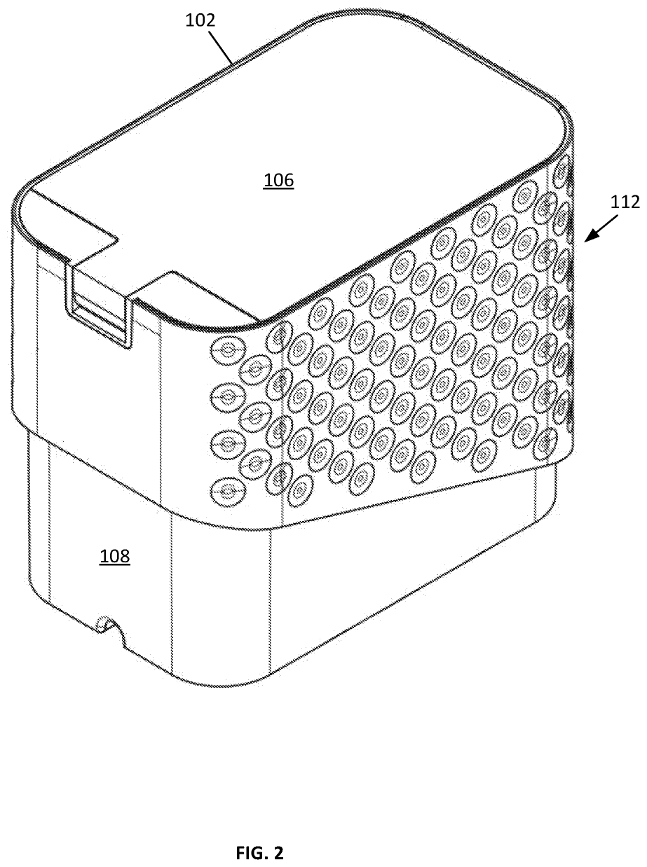

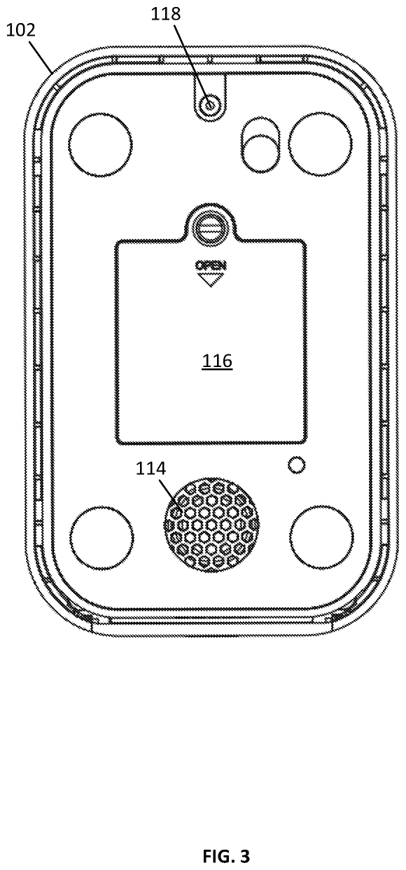

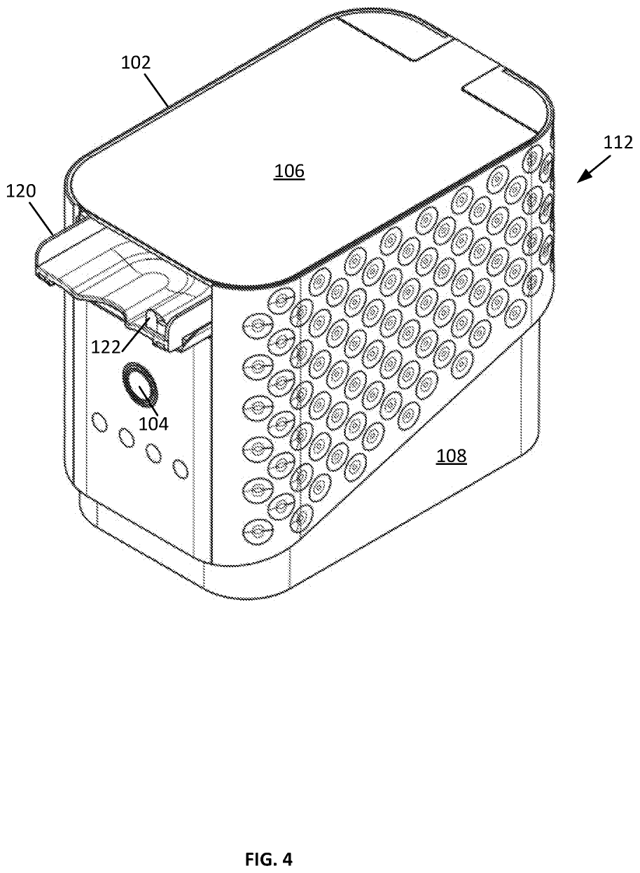

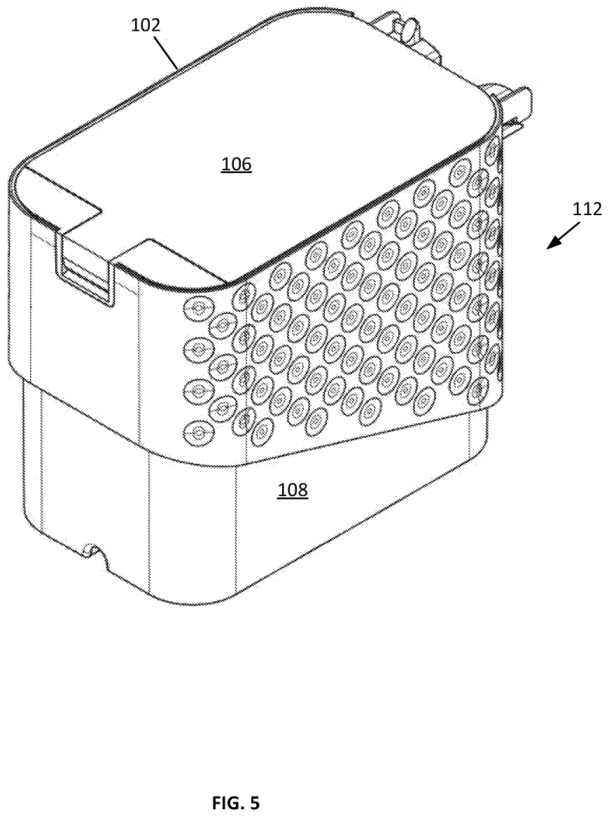

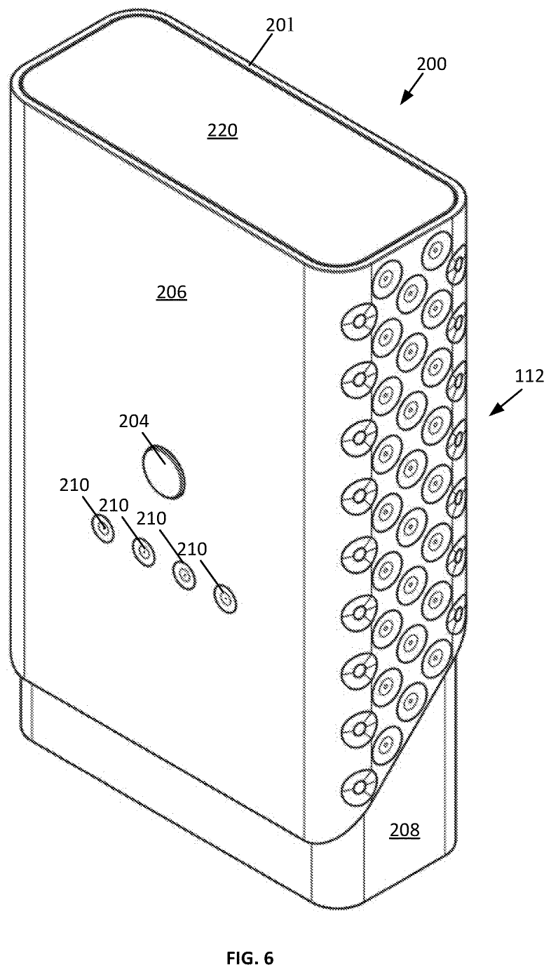

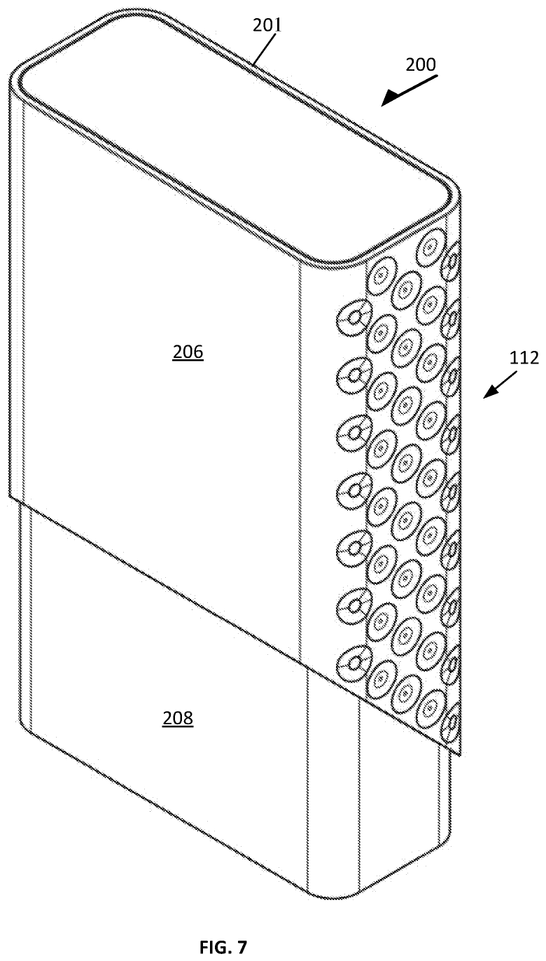

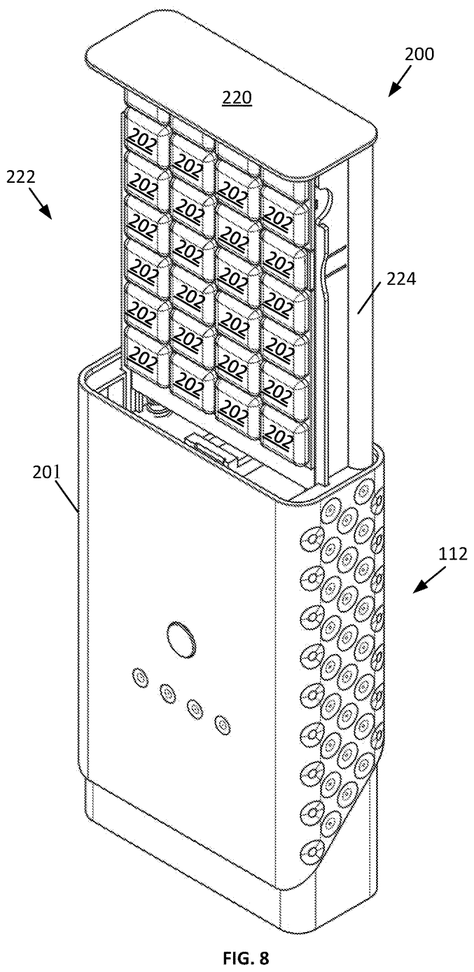

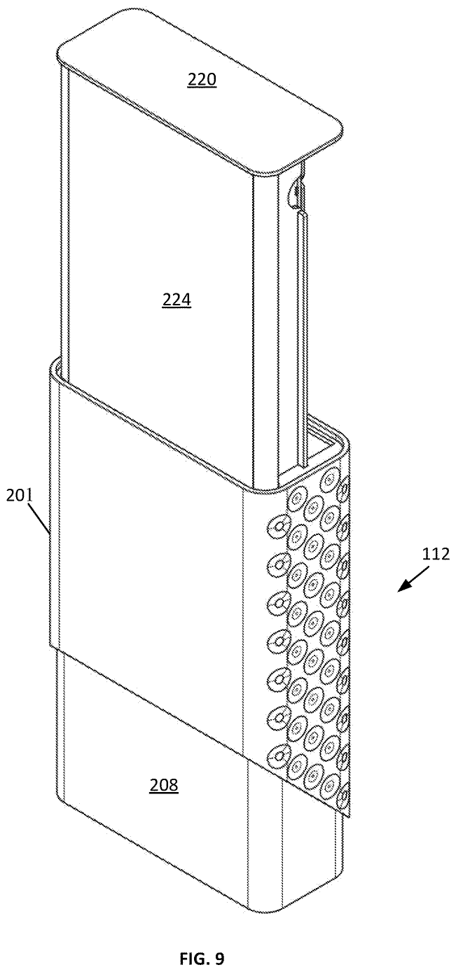

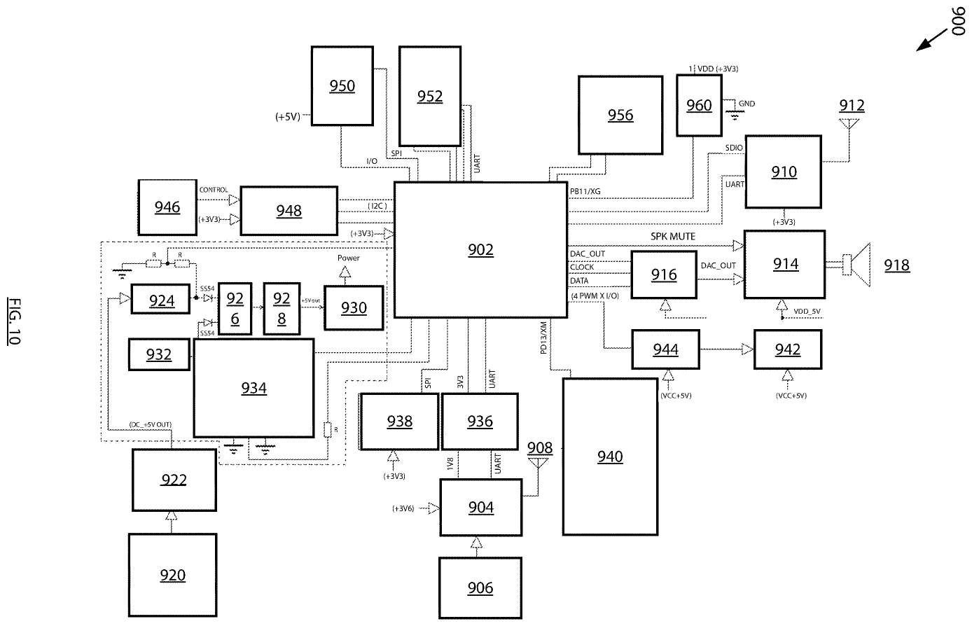

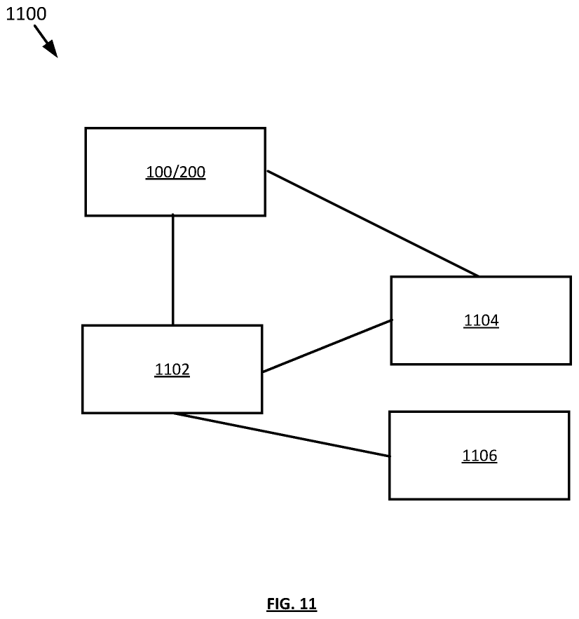

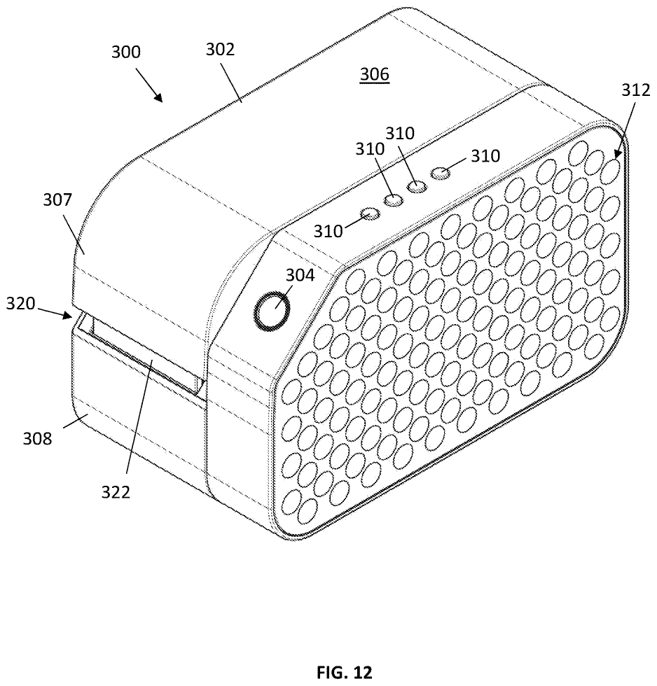

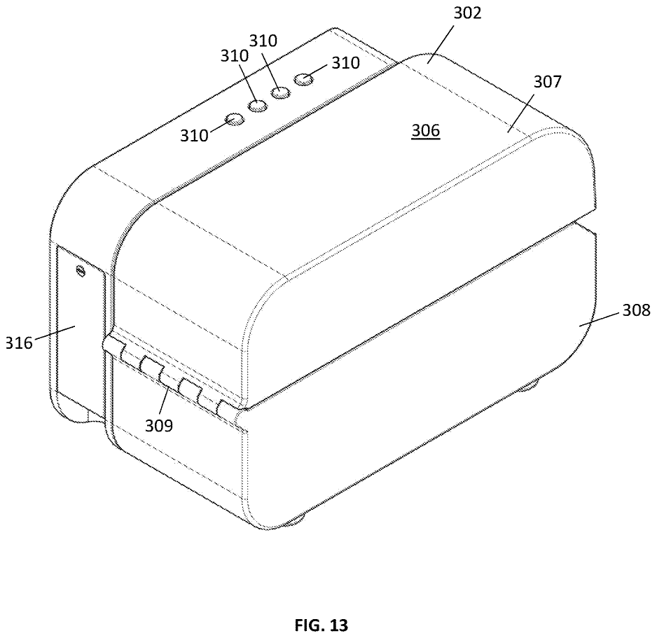

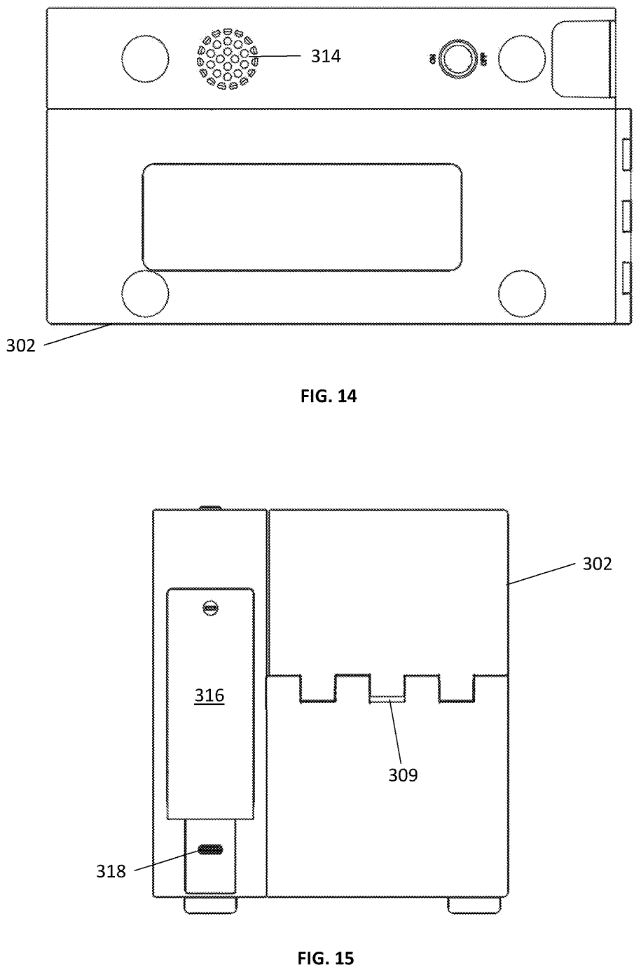

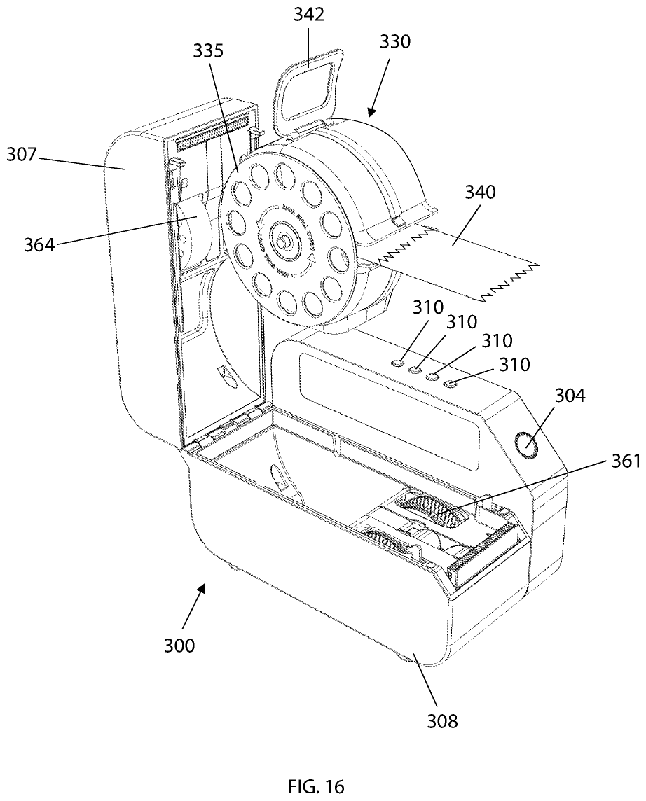

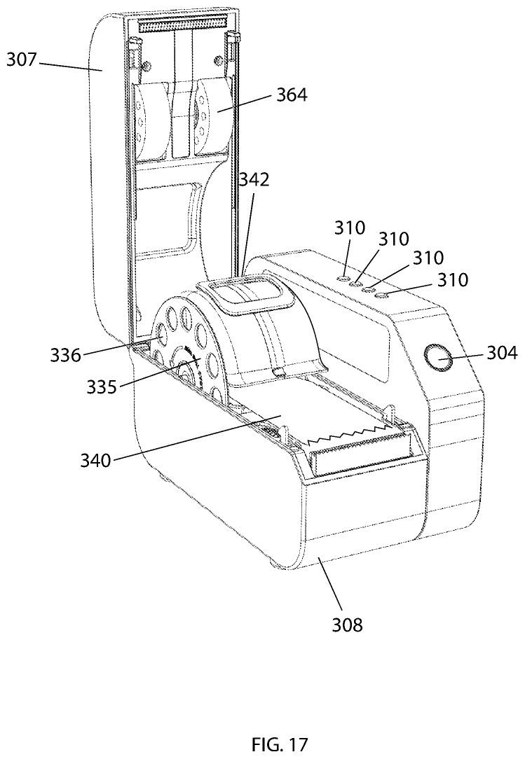

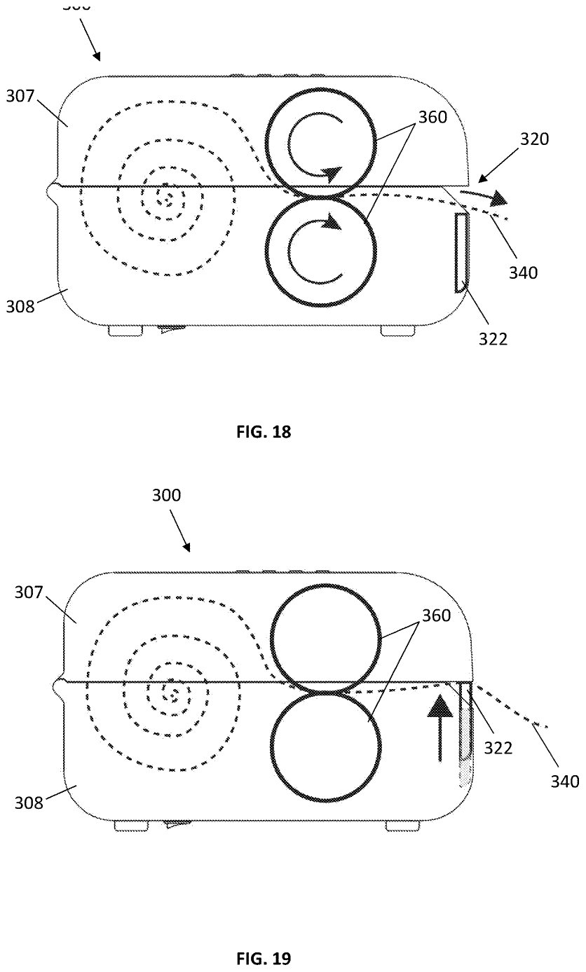

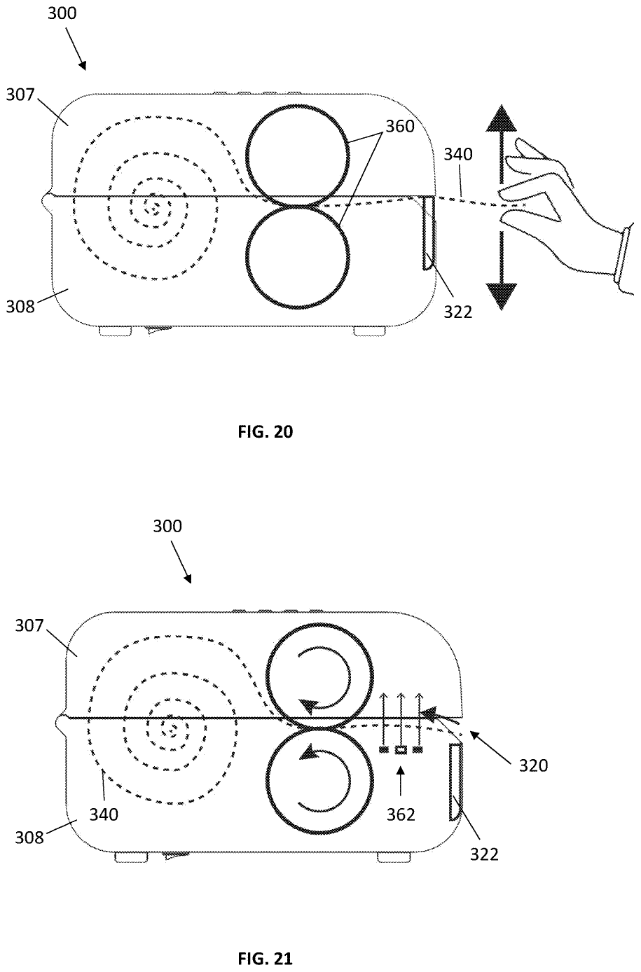

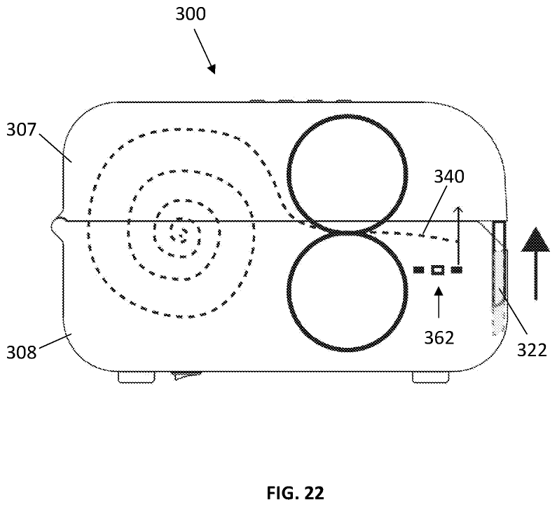

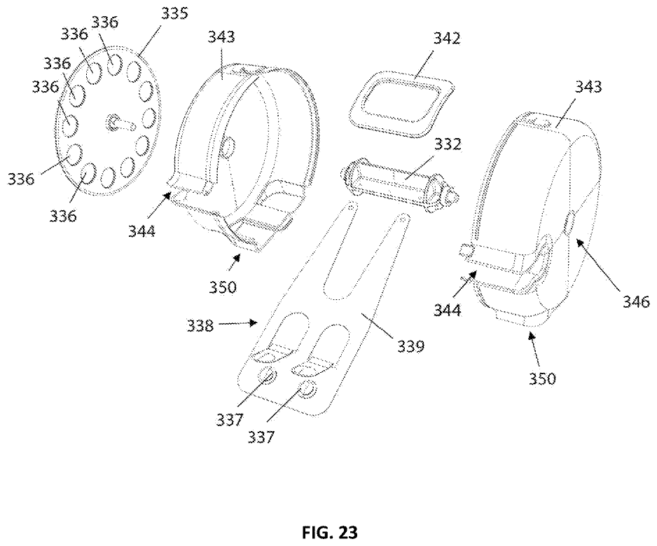

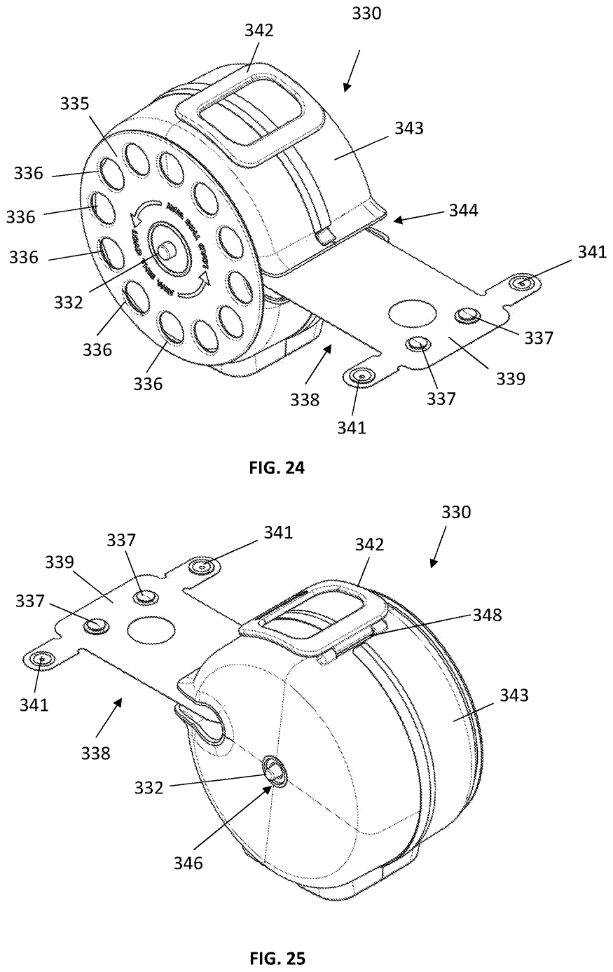

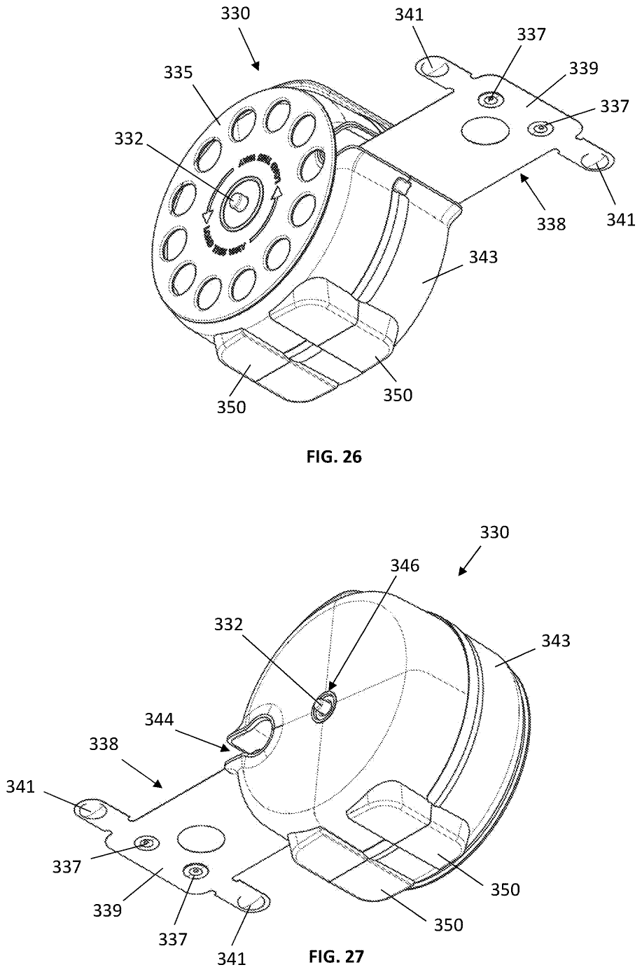

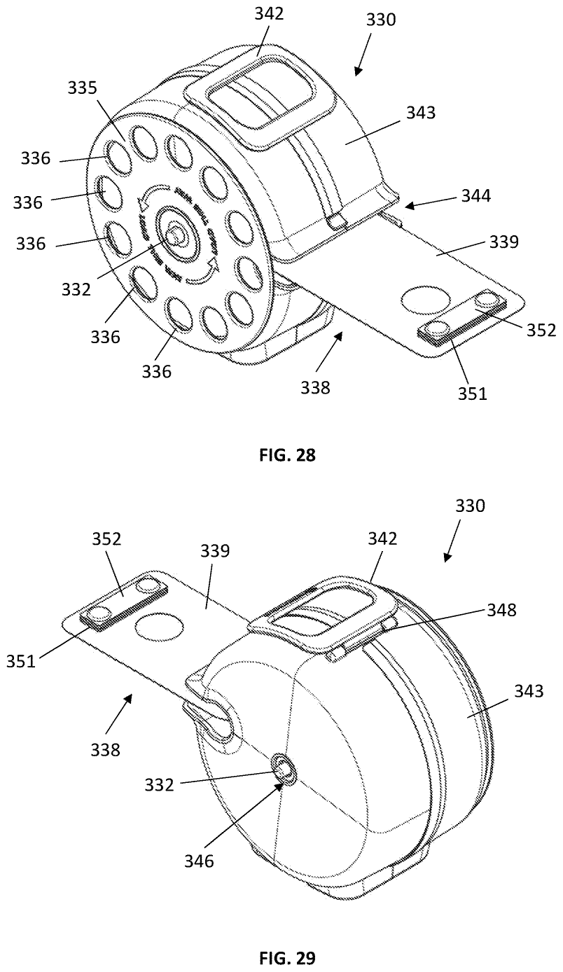

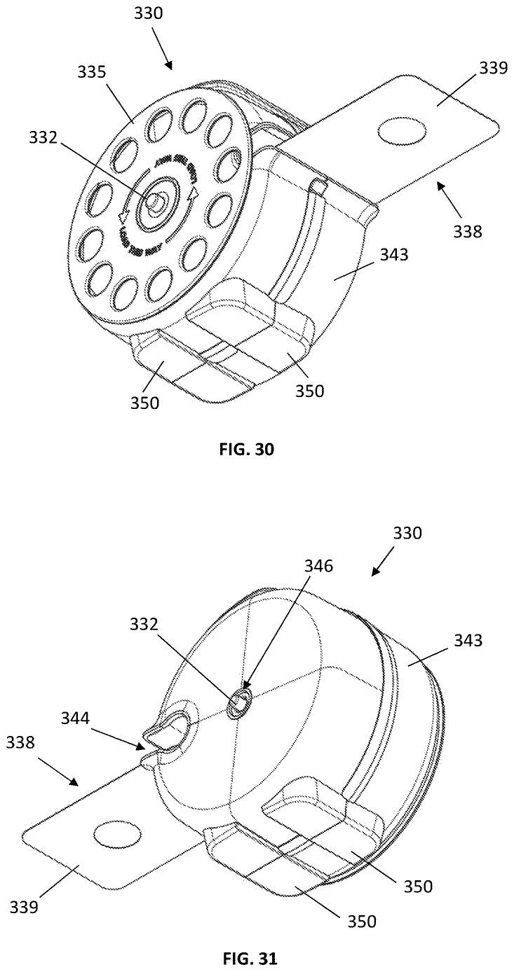

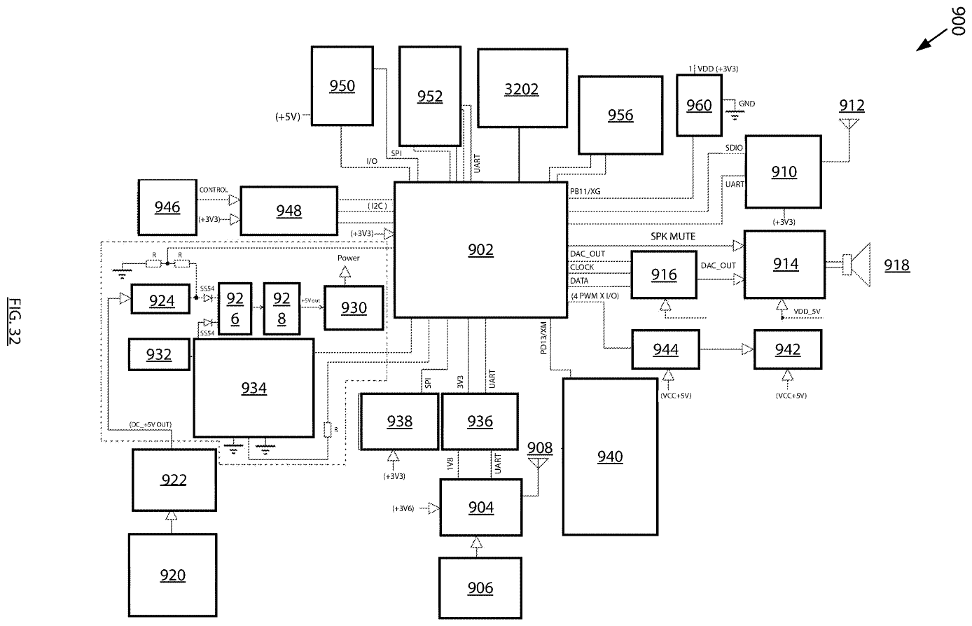

Some embodiments of the present invention are hereinafter described, by way of non-limiting example only, with reference to the accompanying drawings, in which:
a. FIG. 1 is a front perspective view of a first embodiment of an apparatus with a sachet delivery mechanism in a closed condition;
b. FIG. 2 is a rear perspective view of the apparatus of FIG. 1 in the closed condition;
c. FIG. 3 is an underside view of the apparatus of FIG. 1;
d. FIG. 4 is a front perspective view of the apparatus of FIG. 1 in an open condition;
e. FIG. 5 is a rear perspective view of the apparatus of FIG. 1 in the open condition;
f. FIG. 6 is a front perspective view of an apparatus with a container delivery mechanism in a closed condition;
g. FIG. 7 is a rear perspective view of the apparatus of FIG. 6 in the closed condition;
h. FIG. 8 is a front perspective view of the apparatus of FIG. 6 in an open condition;
i. FIG. 9 is a rear perspective view of the apparatus of FIG. 6 in the open condition;
j. FIG. 10 is a block diagram of an electronic circuit of the apparatus of FIG. 1 or 6;
k. FIG. 11 is a block diagram of a system including the apparatus of FIG. 1 or 6;
l. FIG. 12 is a front perspective view of a second embodiment of an apparatus with a sachet delivery mechanism in a closed condition;
m. FIG. 13 is a rear perspective view of the apparatus of FIG. 12 in the closed condition;
n. FIG. 14 is an underside view of the apparatus of FIG. 12;
o. FIG. 15 is a rear view of the apparatus of FIG. 12;
p. FIG. 16 is a front perspective view of the apparatus of FIG. 12 with an upper portion of an outer shell in an open position and a cartridge being inserted into/removed from the apparatus;
q. FIG. 17 is a front perspective view of the apparatus of FIG. 12 with the upper portion of the outer shell in the open position and the cartridge located in an administration/delivery position in the apparatus;
r. FIG. 18 is a schematic side view of the apparatus of FIG. 12 with the sachet delivery mechanism in an open position;
s. FIG. 19 is a schematic side view of the apparatus of FIG. 12 with the sachet delivery mechanism in a closed position;
t. FIG. 20 is a schematic side view of the apparatus of FIG. 12 with the sachet delivery system in a closed position and a user tearing/removing a sachet;
u. FIG. 21 is a schematic side view of the apparatus of FIG. 12 illustrating sensors sensing a position of a sachet; and
v. FIG. 22 is a schematic side view of the apparatus of FIG. 12 with the sachet delivery system in a closed position and the sachets in a pre-defined starting position.
w. FIG. 23 is an exploded view of an embodiment of a cartridge for use with the apparatus of FIG. 1 or 12;
FIG. 24 is a front perspective view from above of the cartridge of FIG. 23;
y. FIG. 25 is a rear perspective view from above of the cartridge of FIG. 23;
z. FIG. 26 is a front perspective view from below of the cartridge of FIG. 23;
aa. FIG. 27 is a rear perspective view from below of the cartridge of FIG. 23;
bb. FIG. 28 is a front perspective view from above of another embodiment of a cartridge;
cc. FIG. 29 is a is a rear perspective view from above of the cartridge in FIG. 28;
dd. FIG. 30 is a front perspective view from below of the cartridge of FIG. 28;
ee. FIG. 31 is a rear perspective view from below of the cartridge of FIG. 28; and
ff. FIG. 32 is a block diagram of an electronic circuit of the apparatus of FIG. 1 or 6 or 12 with an RFID sensor.

### DETAILED DESCRIPTION

### Overview

Described herein is an apparatus (medication administration/delivery device) that is configured and used for administering/delivering medication/pills/medicaments in containers/packages.

Each container/package of the apparatus is configured to include multiple pills/medicaments per dose (including oral medications/pills/medicaments).

A first embodiment of the apparatus includes:
a. a solid/water/tamper-resistant housing for containing/protecting/storing a plurality of the containers/packages containing the pills/medicaments;
b. a power source, which may include an electrical battery and/or an electrical supply connection, in the housing;
c. an actuator arrangement (which may include at least one solenoid and/or at least one (stepper) motor) powered by the power source and configured to administer/deliver the pills/medicaments for a pre-selected patient (e.g., by unlocking/releasing the housing and/or by moving the containers/packages from inside the housing); and
d. at least one sensor (which includes an optical biometric sensor, a touch/fingerprint biometric sensor, and/or a radio-frequency identification (RFID) sensor) powered by the power source to activate the actuator arrangement to allow the at least one package to be opened/accessed manually (e.g., by unlocking/releasing the housing and/or by moving the containers/packages from inside the housing) when the sensor detects a pre-defined identifier (ID) of the pre-selected patient.

The sensor may include a biometric sensor that may include a fingerprint scanner and/or a camera/optical scanner, and the ID may be biometric ID and may include respectively a fingerprint and/or a face of the patient. The at least one sensor may include a radio-frequency identification (RFID) sensor and the pre-defined identifier may be an RFID identifier. The RFID sensor may include an RFID reader configured to read a tag that includes the pre-defined identifier (ID), in or on a fob carried by a person, typically the pre-selected patient. The at least one sensor may include a plurality of sensors, including a biometric sensor and a radio-frequency identification (RFID) sensor, for example, and the sensors may activate the actuator arrangement when only one, or only when all, of the sensors detect respective pre-defined identification of the patient.

A second embodiment of the apparatus includes:
a. a housing for containing/protecting/storing a plurality of interconnected sachets containing pills/medicaments;
b. the actuator arrangement including a pair of counter-rotating wheels mounted within the housing configured to administer/deliver the sachets containing pills/medicaments (the wheels forming part of the actuator arrangement for moving the containers/packages from inside the housing);
c. at least one microcontroller unit (MCU) configured to control a motor of the actuator arrangement coupled to one of the wheels; and
d. a plurality of sensors disposed within the housing, the plurality of sensors including at least one of an optical sensor and a motion sensor, wherein the sensors are configured to detect an edge of a leading one of the sachets and communicate with the MCU to stop the motor when the sachets are in a starting position defined by a location of the leading edge relative to the actuator arrangement or the housing.

Also described herein is a cartridge including:
a. an axle;
b. a lever configured to hold/turn the axle and connected to the axle when in use; and
c. a gripper attached to the axle and configured to grip an edge of a first sachet in a strip of interconnected sachets containing medication/medicament to wind the strip of interconnected sachets onto the axle by holding/turning the lever.

The second embodiment can also include the features of the first embodiment, including:
a. the solid/water/tamper-resistant housing for containing/protecting/storing a plurality of the containers/packages containing the pills/medicaments;
b. the power source, which may include an electrical battery and/or an electrical supply connection, in the housing;
c. the actuator arrangement (which may include at least one solenoid and/or at least one (stepper) motor) powered by the power source and configured to administer/deliver the pills/medicaments (e.g., by unlocking/releasing the housing and/or by moving the containers/packages from inside the housing) for a pre-selected patient (i.e., a person); and
d. the at least one sensor powered by the power source to activate the actuator arrangement to allow the at least one package to be opened/accessed manually (e.g., by unlocking/releasing the housing and/or by moving the containers/packages from inside the housing) when the sensor detects a pre-defined identifier (ID) of the pre-selected patient.

### Implementations

As shown in FIG. 1, a first embodiment of the apparatus may be in the form of a sachet delivery apparatus 100 that is configured and used for administering/delivering the pills/medicaments in the packages, with its housing 102 having dimensions (in mm) of substantially 170L×120W×180H. The housing 102 may be sufficiently large to contain 28 packages (for a month of use), or a sachet roll of substantially 110-mm diameter, e.g., including a pre-prepared roll of DAA packages. Each package is pre-filled with pre-selected pills for a day based on the patient medical record. As shown in FIG. 4, the apparatus may include a reversibly extendable shelf 120 that moves to a projecting condition, in which it projects outward from a side of the housing 102, to present a tray/cup depression configured to hold the pills/sachet that is being administered/delivered, thus configured to hold one sachet in a condition/location relative to the housing such that the sachet can be easily manually grasped by the patient. The actuator arrangement, e.g., the stepper motor, can move/push/drive the shelf 120 out such that the pills are visible/accessible when the actuator arrangement is activated. As shown in FIG. 4, the apparatus may include a projection/tooth/pin 122 that is solid and at least partially sharp to assist with tearing, and or sufficiently sharp to tear, the package/sachet from its roll (thus from its adjacent package/sachet in the roll): the pin 122 may be mounted/arranged/formed on the shelf 120 where the administered/delivered pills/sachet is held on the shelf 120, e.g., on a side of the shelf 120 where the pin 122 is located/shaped to naturally perforate an edge/side of the package/sachet when pressed into/onto the package/sachet, e.g., where perforations may already be pre-formed in the roll between packaged/sachets. The pin 122 may make it easier for the patient, who may have reduced hand strength, to detach the administered/delivered package/sachet from the roll without requiring a separate mechanism (which could add cost/complexity/expense). The pin 122 may be formed of the same material as the shelf, e.g., injection moulded plastic.

As shown in FIG. 6, the apparatus may be in the form of a (non-sachet) container delivery apparatus 200 that is configured and used for administering/delivering the pills/medicaments in separated containers (rather than requiring sachets). The container delivery apparatus 200 may have a housing 201 with dimensions (in mm) of substantially 110L×55W×182H for 14 containers (e.g., a 2 by 7 grid of the containers), or substantially 130L×55W×207H for 28 containers (e.g., a 4 by 7 grid of the containers). The container delivery apparatus 200 may be manually portable, e.g., easy for an adult to carry in one hand and/or in a handbag or laptop bag, e.g., more portable than the sachet delivery apparatus 100. The top face of the delivery apparatus 200 may include a cartridge draw lid 220 that pushes out from the housing to reveal/expose the containers 222 in a reversibly openable cartridge drawer 224, e.g., as shown in FIGS. 6 and 8. The actuator arrangement may include a solenoid or motor that releases/unlocks the cartridge drawer 224, allowing it to be manually pulled open, and/or allowing it to be pushed/pulled open by a spring mechanism, e.g., one or more springs in the apparatus that push/pull the drawer 224 along tracks in the apparatus towards its open condition shown in FIGS. 8 and 9 (from the closed condition shown in FIGS. 6 and 7), thus allowing manual access to at least one of the containers, specifically to the container selected by the apparatus based on its stored schedule and pill dose due on that day for that patient.

As shown in FIG. 8, the containers are sealed/closed by respective lids 202 that are individually opened to access the containers (and the pills therein), and each container is pre-filled with pre-selected pills for a day based on the patient medical record.

The housing includes an outer shell that is solid, water resistant to keep the pills dry, and tamper resistant to keep the pills secure until they are administered/delivered according to the schedule. The outer shell may be formed of plastic. The outer shell 106,206, as shown in FIGS. 1 and 6, may fit over a main base body 108,208, that may be configured to rest on a table/bench. The main base body may also be solid, water resistant to keep the pills dry, and tamper resistant to keep the pills secure, and may be formed of plastic. The outer shell 106,206, as shown in FIGS. 1 and 6, may include at least one face including a grid/pattern of dimples/divots 112,212 formed in the surface of the outer shell, e.g., on a left side and an opposed right side.

The fingerprint scanner may be substantially circular, mounted substantially centrally on one face of the apparatus, e.g., on a front face as shown in FIG. 1 (as fingerprint scanner 104) or on a front face as shown in FIG. 6 (as fingerprint scanner 204).

The apparatus may include an audio/visual (A/V) system, including an audio speaker and/or a light/display in/on the housing, configured to play/display alerts (local alerts) for the patient. The light/display may include one or more optical indicators, e.g., lights or light-emitting diodes, configured/arranged to be visible to the patient when the apparatus is a normal standing orientation, standing on the main base body. The optical indicators may include 1 to 4 optical indicators located in the outer shell, on the front face as shown in FIG. 1 (as buttons/indicators 110) or on the front face as shown in FIG. 6 (as buttons/indicators 210). The audio speaker may be mounted/arranged in the main base body, with an acoustic outlet in the main base body, e.g., perforations/holes, e.g., in the form of a speaker outlet 114 as shown in FIG. 3.

The apparatus may include a user interface (UI) for a user or the patient to control the apparatus and its system. The UI can include a plurality of control buttons and/or a touch screen. The control buttons are mounted/arranged in the housing, e.g., in the outer shell, e.g., on a top face or side face of the outer shell. The control buttons may include 1 to 4 control buttons located in the outer shell, on the front face as shown in FIG. 1 (as buttons/indicators 110) or on the front face as shown in FIG. 6 (as buttons/indicators 210). The control buttons may be capacitive touch buttons. The touch screen may be a capacitive touch screen.

The apparatus may include one or more printed circuit board assemblies (PCBAs) arranged/mounted in the main base body, e.g., adjacent and/or mounted to the floor of the main base body. The power source may be arranged/mounted adjacent and/or mounted to the floor of the main base body for improved stability/safety.

The housing may include a socket 118 for connection of the electrical supply connection, e.g., to a mains power supply. The housing may include a manually removable cover 116 configured to secure releasably the electrical battery. The socket 118 and the cover 116 may be arranged in the main base body, e.g., on the bottom face as shown in FIG. 3.

The apparatus includes one or more electronic circuits 900 (e.g., in PCBAs) with an electronic controller in the form of at least one microcontroller unit (MCU) 902, e.g., as shown in FIG. 10. The MCU is configured to control the actuator/s (to activate the actuator arrangement to unlock or release the housing) based on the at least one sensor detecting the pre-defined identifier of the pre-selected patient.

As shown in FIG. 10, the electronic circuits 900 may also include at least one network interface module/card (“network interface”) configured to allow electronic/data communication between the MCU and the remote computing system, including via Cellular/Bluetooth/WiFi/Ethernet/Internet protocols, e.g., including Ethernet (10/100 Mbit), WiFi (2.4 and 5 GHz), Cellular (including LTE, Cat-M, and NB IoT), Serial/UART, Zigbee/Z-Wave, LoRa, Modbus, and/or Ethernet/IP. The network interface may include a cellular module 904, a SIM card 906, a translator module 936 (connected to the MCU 902) and an antenna 908 (connected to the cellular module 904) configured to connect to a commercial cellular network, e.g., a 4G or a 5G network. The network interface may include a WiFi and/or Bluetooth module 910 and a corresponding WiFi/BT antenna 912 to connect to a WiFi network and/or BT device.

As shown in FIG. 10, the electronic circuits 900 may include an audio amplifier 914 and audio control module 916, driven by the MCU 902, and configured to power the speaker 918.

As shown in FIG. 10, the electronic circuits 900 may include the power source including: a DC power supply 920 configured to receive a mains power supply, and powering a protection module 922. The supply 920 powers an input circuit 924 that can power the other components in the electronic circuits 900 via a switch 926, regulator 928 and power management module 930. The power source may also include at least one battery 932, e.g., multiple AA batteries, monitored by the MCU 902 via a monitoring module 934, also able to power the other components in the electronic circuits 900 via the switch 926 (which can select between the mains and the battery 932), the regulator 928 and the power management module 930.

As shown in FIG. 10, the electronic circuits 900 may include a position detector switch 940 that is arranged in the apparatus to detect/monitor whether the housing has been unlocked/released, that is, whether the housing is open to administer/deliver and allow manual picking of the pills/medicaments. The switch 940 may detect the open/extended condition of the shelf 120, or the open condition of the drawer 224.

As shown in FIG. 10, the electronic circuits 900 may include:
a. the motor in the form of a stepper motor 942 controlled by a driver 944 that is in turn controlled by the MCU 902;
b. the buttons/indicators 110 in the form of touch keys 946 and a touch switch 948 (which can power the apparatus on and off) that are both connected to the MCU 902;
c. the buttons/indicators 110 in the form of timer LED indicators 950 and/or status LEDs 956 controlled by the MCU 902;
d. the at least one sensor in the form of an electronic biometric sensor 952 powered by the power source and connected to the MCU 902 to send sensed patterns/signals to the MCU 902 to indicate the sensed biometric quantities; and
e. a lid switch 960 configured/arranged to detect when a lid of the housing is open, which is typically to fill the pills/medicaments, and connected to the MCU 902 to keep the MCU 902 in a fill/refill state.

The MCU 902 may be configured to perform a biometric setup process, including receiving a user input from the biometric sensor representing a biometric identifier (input biometric identifier) of the user/patient, and storing the input biometric identifier as the pre-defined biometric identifier.

The MCU 902 may be configured to control the A/V system based on a calendar/clock and a preselected schedule stored in the MCU 902 and/or accessed by the MCU 902 from a remote computing system, which may include cloud servers, wherein the preselected schedule defines reminders to administer/deliver the pills/medicaments based on the preselected schedule (from the patient medication record).

The MCU 902 may be configured to access a patient medication schedule (e.g. including timing and quantity for a given dose) for the pre-selected patient (stored in the MCU and/or accessed on the remote computing system), wherein the biometric sensor is configured to activate only when sensing the biometric identifier (visible or touch) of the patient, wherein the patient medication schedule is provided/sent by a medical provider (pharmacy) that also provides/dispenses the medication in the containers/packages according to the patient medication schedule.

The MCU 902 may be configured to send (remote) alerts to the remote computing system for distribution to selected user communication devices (e.g., mobile devices of carers with contact identifiers recorded in the MCU and/or the remote computing system).

The MCU 902 may receive the patient medication schedule, and/or machine-readable routines to control its operation, via machine-readable memory, e.g., in the MCU 902 and/or in a memory module 938.

As shown in FIG. 11, the apparatus, e.g., 100/200, may be part of a system 1100 that includes one or more of the following:
a. a remote computing system 1102 with at least one recording/reporting module configured to (i) receive and store usage data from the apparatus representing records of the dispensing of the pills/medicaments (including which packages/containers and corresponding dates/times from the calendar/clock), and (ii) generate reports based on the stored records, including reports on adherence, for access by care institutions, doctors and pharmacists;
b. an application (app) on a handheld smart device 1104 (e.g., smart phone) and/or a personal computer (PC) configured to communicate with the remote computing system to receive the remote alerts, and to generate A/V alerts using the display/lights/speaker of the handheld smart device and/or PC to alert a remote user of the handheld smart device/PC (e.g., a carer);
c. an application on a computing system 1106 controlled by a medical provider (e.g., pharmacy, doctor, care institution), wherein the computing system 1106 is configured/permitted to access private data of the patient including the patient medication record or the patient medication schedule, wherein the application is configured to receive usage data from the apparatus and the reports from the remote computing system, wherein the usage data include records of operation of the at least one sensor to activate the actuator arrangement (identified by patent ID), and/or records of the medication being administered/delivered (identified by patent ID), wherein the records include recorded dose identifiers and respective times/dates of administration/delivery from the apparatus, wherein the application is configured to match the records with the patient medication schedule (using the patent ID); and
d. electronic/data communication links connecting the elements of the system 1100, including BT links from the apparatus 100/200 to the smart device 1104, cellular/WiFi and Internet connections between the apparatus 100/200 and the remote system 1102, and Internet connections between the remote system 1102 and both the smart device 1104 and the provider's computing system 1106.

As shown in FIG. 12, a second embodiment of the apparatus may be in the form of a sachet delivery apparatus 300 that is configured and used for administering/delivering the pills/medicaments in the packages. The apparatus includes a housing 302 for containing/protecting/storying multiple interconnected packages, or interconnected sachets 340, containing the pills/medicaments, in a sachet roll, e.g., a prepared roll of DAA packages. The housing 302 includes an outer shell 306 having an upper portion 307 and a lower portion 308. The housing dimensions (mm) may be substantially 250L×140W×160H, or preferably 252L×136W×156H.

As shown in FIGS. 13 and 15, the housing 302 may include a socket 318 for connection of the electrical supply connection, e.g., to a mains power supply. The housing 302 may include a manually removable cover 316 configured to secure releasably the electrical battery. The socket 318 and the cover 316 may be arranged in the main base body, e.g., on the rear face as shown in FIG. 15.

The housing 302 includes an outer shell that is solid, water resistant to keep the pills dry, and tamper resistant to keep the pills secure until they are administered/delivered according to the schedule. The outer shell 306 may be formed of plastic. The outer shell 306, as shown in FIG. 12, may include at least one face including a grid/pattern of dimples/divots 312 formed in the surface of the outer shell, e.g., on a left side or a right side.

The fingerprint scanner may be substantially circular, mounted substantially centrally on one face of the apparatus, e.g., on a 45-degree-angled face of forward facing portion as shown in FIG. 12 (as fingerprint scanner 304).

The apparatus may include an audio/visual (A/V) system, including an audio speaker and/or a light/display in/on the housing, configured to play/display alerts (local alerts) for the patient. The light/display may include one or more optical indicators, e.g., lights or light-emitting diodes, configured/arranged to be visible to the patient when the apparatus is a normal standing orientation, standing on the main base body. The optical indicators may include a plurality of optical indicators located in the outer shell, on an upper face as shown in FIG. 12 (as buttons/indicators 310). The audio speaker may be mounted/arranged in the main base body, with an acoustic outlet in the main base body, e.g., perforations/holes, e.g., in the form of a speaker outlet 314 as shown in FIG. 14.

The apparatus may include a user interface (UI) for a user or the patient to control the apparatus and its system. The UI can include a plurality of control buttons and/or a touch screen. The control buttons are mounted/arranged in the housing, e.g., in the outer shell, e.g., on a top face or side face of the outer shell. The control buttons may include 1 to 4 control buttons located in the outer shell, on the front face as shown in FIG. 12 (as buttons/indicators 310). The control buttons may be capacitive touch buttons. The control buttons may alternatively or additionally be tactile buttons that click when pressed. The touch screen may be a capacitive touch screen.

As shown in FIG. 13, the housing 302 may include a hinge 309 to allow the upper portion 307 to pivot relative to the lower portion 308. FIGS. 16 and 17 show the upper portion 307 in an open position. In the open position, a user/patient can access an internal area for containing/protecting/storying multiple interconnected packages, or sachets 340.

As shown in FIGS. 17 to 22, the actuator arrangement of the apparatus 300 may include a pair of counter-rotating wheels 360 mounted within the housing. The wheels are configured to administer/deliver the sachets containing pills/medicaments. The counter-rotating wheels 360 in the pair may be biased towards one another in order to grip the interconnected sachets 340 therebetween. A spring, or springs, may be used to provide the biasing means. One wheel in the pair of wheels may include gripping material 361 (e.g., material with a high coefficient of static friction) to grip the interconnected sachets. The gripping material may be a silicone rubber, for example, and include spikes/projections to increase the coefficient of friction. One wheel in the pair of wheels may include deformable material 364 (e.g., resilient material) that deforms around the pills/medicaments in the interconnected sachets. The deformable material may be a silicone sponge, for example, and have a substantially smooth surface.

The wheels may be configured to distribute pills/medicaments in the interconnected sachets 340, e.g., when the pills/medicaments are mutually stacked in the interconnected sachets. For example, if a pill is stacked on top of another pill in the sachet, as the sachet passes between the counter-rotating wheels, after being deformed by the additional thickness of the stacked pills, the deformable material urges the pills into a single layer. In other words, the wheels may act to disperse the pills more evenly throughout the sachet.

The apparatus 300 may also include at least one microcontroller unit (MCU) configured to control a motor of the actuator arrangement coupled to one of the wheels, e.g., the MCU 902. The motor may be configured to have a speed and torque sufficient to drive the wheels to administer/deliver the sachets. The MCU may be configured to control the motor based on a preselected administration/delivery length stored in or accessed by the MCU. The administration/delivery length is typically similar to the length of each individual sachet, which varies between different brands/providers of packaged pills/medicaments. For example, sachets from one brand might be 75 mm long, where sachets from another brand might be 80 mm long. The number of revolutions of the counter-rotating wheels required to administer/deliver a sachet is approximately equal to the circumference of the wheel that is connected to the motor divided by the length of each individual sachet. The administration/delivery length may be preselected by an operator when on-boarding a new user/patient. Alternatively, the administration/delivery length may be selected by the user/patient.

As shown in FIGS. 16 and 17, the apparatus may include two pairs of counter-rotating wheels. In embodiments that include two pairs of wheels, one wheel from each pair (i.e. two wheels) is rotatably mounted in the upper portion 307 of the housing, and the remaining wheel from each pair is rotatably mounted in the lower portion 308 of the housing. Embodiments with two pairs of counter-rotating wheels more reliably direct the sachets in a straight path during administration/delivery when compared to embodiments with one pair of counter-rotating wheels.

The apparatus 300 may also include a sensor arrangement 362 in the form of a non-contact sensor arrangement that detects the sachets without hindering their movement. The sensor arrangement may include a plurality of (non-contact) sensors disposed within the housing 302. The plurality of sensors may include at least one of an optical sensor and a motion sensor. The sensors may be configured to detect an edge of a leading one of the sachets and communicate with the MCU to stop the motor when the sachets are in a starting position defined by a location of the leading edge relative to the wheels. The starting position is illustrated in FIG. 22. The plurality of sensors may include two optical interrupters and one motion sensor, with the motion sensor arranged between the optical interrupters such that the edge of the sachet passes in the following order one optical interrupter, then the motion sensor, then the other optical interrupter. The sensor arrangement 362 provides reliable recognition of the edge of a sachet.

As shown in FIGS. 12 and 18 to 22, the apparatus 300 may include an aperture 320 (“administration/delivery aperture”) through which the sachets can be administered/delivered by the wheels. The aperture 320 is larger in width and height than the sachets that pass through the aperture 320, and is smaller in at least one of width and height than a person's finger, or at least a person's hand, thereby to resist a person manually accessing the sachets through the aperture 320, at least when the sachets are interior of the starting position. The apparatus 300 may also include a lockout clamp 322, or gate, movable between an open position (as shown in FIGS. 18 and 21), which opens the aperture 320, and a closed position (as shown in FIGS. 19, 20 and 22), which closes the aperture 320 to block the interconnected sachets from moving through the aperture 320. The lockout clamp 322 provides increased security and ensures that a user/patient cannot access more pills/medicaments in sachets that are not included in a given prescribed dose.

As shown in FIGS. 23 to 27, a cartridge 330 may be provided for use in a medication administration/delivery apparatus that administers/delivers medication in containers/packages/sachets, with each container/package/sachet configured to include multiple pills/medicaments per dose (including oral medications). The cartridge 330 may include an axle 332, a lever configured to hold/turn the axle 332 and connected to the axle when in use, and a gripper 338 attached to the axle 332 and configured to grip an edge of a first containers/packages/sachet in a strip of interconnected containers/packages/sachets containing medication/medicament to wind the strip of interconnected containers/packages/sachets onto the axle 332 by holding/turning the lever. The axle 332 may have a diameter of between 10 mm and 15 mm. The gripper 338 may also be referred to as a “tensile member” because it acts to pull the sachets as the sachets are wound onto the axle 332. The cartridge provides a convenient means for medication dispensing pharmacies to package and store medications for a patient. Each cartridge may be designed to store a patient's medication requirements for a given period—one week or one month, for example. The number of sachets that can be stored on a cartridge may be dependent upon the number and size of pills required in each sachet.

The gripper 338 may include a flexible tongue 339 and/or a fastener (e.g., press studs or a magnet). The fastener may deform/perforate the edge of the first sachet in order to grip the sachet. For example, the gripper 338 can include press studs that have a male portion 337 and female portion 341, as shown in FIGS. 24 to 27. The press studs may be attached and detached manually by a person. In some embodiments, the gripper 338 may include at least one magnetic element arranged to grip the sachet. For example, as shown in FIGS. 28 to 31, the gripper 338 may include a first magnetic element 351 connected to the tongue 339, and a second magnetic element 352 (indirectly) couplable to the first magnetic element 351. As shown in FIGS. 28 and 29, the second magnetic element 352 is attracted to the first magnetic element 351 by a magnetic/attraction force that forces the two magnetic elements together, and thus grips the sachet portion when the sachet portion is between the first and second magnetic elements 351, 352. The first and second magnetic elements 351, 352 are configured so that the magnetic/attraction force provides a holding force sufficient to resist removal of the sachet from its engaged position, gripped by the gripper 338. The first magnetic element 351 is fastened to the flexible tongue 339 of the gripper 338 by an adhesive element (e.g., a glue patch) and/or by the magnetic/attraction force when the first magnetic element 351 is fastened to a first side of the and the second magnetic element 352 is attracted to the opposite side of the flexible tongue 339 (thus gripping both the flexible tongue 339 and the end of the sachet when in use). One or both of the first and second magnetic elements 351, 352 includes at least one permanent magnet, e.g., a rare-earth magnet (e.g., two rare earth magnets embedded in a ferromagnetic bar as shown in FIGS. 28 and 29); and one of the first and second magnetic elements 351, 352 can include a ferromagnetic material (e.g., a ferromagnetic bar without embedded magnets). From the engaged position, the magnetic elements can be pulled apart manually by a person in order to remove the sachet. In this way, the gripper 338 is releasably attachable to the strip of interconnected sachets.

The flexible tongue 339 of the gripper 338 may include a portion made of a woven material. The gripper may include a material that is durable, resistant to water, abrasion, bacterial penetration and/or aging. Such a material might include Tyvek.

As shown in FIGS. 23, 24 and 26, the lever may be provided by a disc 335 with a rotational axis aligned with a rotational axis of the axle 332, and being configured for manual holding/turning of the axle 332. The disc 335 may be substantially circular. The disc 335 can also act to guide the strip of interconnected packages/containers/sachets while an operator winds them onto the axle 332. The disc 335 may include two or more apertures 336 (“holding apertures”) disposed circumferentially around the disc 335 for holding/moving the disc 335. The apertures 336 may be configured for gripping manually/by a person's finger. That is, the apertures 336 are sized allow a user to easily rotate the disc to wind and unwind the sachets by locating one or more fingers in the apertures 336. The disc 335 may be removable from the axle 332.

As shown in FIGS. 16, 17 and 23 to 27, the cartridge 330 may include a cartridge housing 343, connected to the axle 332 when in use. The cartridge housing 343 may be configured to: contain the strip of interconnected sachets; and allow administration/delivery of the sachets through an aperture 344 (“cartridge aperture”) in the cartridge housing 343. The cartridge housing 343 may be substantially cylindrical in shape. The cartridge housing 343 may include one or two apertures 346 (“axle apertures”) arranged to: hold one or both ends of the axle 332 that extend beyond the interconnected sachets when on the axle 332; and allow rotation of the axle 332 relative to the cartridge housing 343 to unwind the strip of interconnected sachets from the axle 332. At least one of the ends of the axle 332 may extend though the cartridge housing 343 to meet the lever such that the lever can hold/turn the axle 332 (without holding/turning the cartridge housing 343). The cartridge housing 343 may include two separable portions (e.g., two halves) so that the cartridge housing 343 may be readily assembled and disassembled.

As shown in FIGS. 16, 17 and 23 to 25, the cartridge 330 may include a handle 342 for lifting/lowering/carrying the cartridge 330 when loaded with the strip of interconnected sachets. The handle 342 may include a hinge connection 348 to the cartridge 330 such that the handle 342 can move between: an open condition (as shown in FIG. 16) for the lifting/lowering/carrying; and a closed condition (as shown in FIGS. 17, 24 and 25) for storing/resting the cartridge. The handle 342 may be curved to conform to the curved shape of the outer surface of the cartridge housing 343 when in the closed condition.

As shown in FIGS. 26 and 27, the cartridge 330 may include a foot portion 350 connected to the axle 332 (via the cartridge housing 343) configured to hold the axle 332 substantially parallel to a support surface when the cartridge 330 is resting on the support surface (by gravity, e.g., on a table/bench and/or in a medication administration/delivery machine). The handle 342 may be connected to an outer surface of the cartridge housing 343. The handle 342 may be disposed substantially opposite the foot portion 350.

The cartridge 330 may include a machine-readable identification device configured to be detected/read by an optical/RF detector/reader for uniquely identifying one of a plurality of the cartridges. The identification device may include an optically readable label (e.g., a barcode or QR code) and/or an RF readable tag (e.g., an RFID tag). The identification device may include information such as the name of the patient for whom the medications are intended, the medications that have been packed onto the cartridge, and where the cartridge was packed. In some embodiments, a user/patient will be supplied with two cartridges at a time to prevent being without medication when the pills/medicaments from the first cartridge have been fully administered/delivered. The cartridge 330 may be reusable.

As illustrated in FIGS. 18 to 22, the apparatus, e.g., 300, may be part of a process that includes one or more of the following:
a. powering the actuator arrangement, including a first actuator, to move at least one of a plurality of interconnected sachets stored within a housing towards an aperture in the housing, and/or powering the actuator arrangement, including the first actuator, to move the plurality of interconnected sachets away from the aperture in the housing;
b. sensing, with a plurality of sensors, when the plurality of interconnected sachets is in a pre-defined starting position; and
c. restricting manual access to the interconnected sachets stored within the housing.

The process may also include activating the actuator arrangement, e.g., a second actuator that is separate from the first actuator, to unlock or release at least one of the interconnected sachets when the at least one sensor detects the pre-defined identifier of the pre-selected patient/person. In this embodiment, the at least one sensor may include the biometric sensor (e.g., the fingerprint scanner 304) and/or an RFID sensor. The first actuator may include one wheel, or a pair of counter-rotating wheels, and at least one motor configured to drive the wheel/s. The second actuator may include a solenoid and/or linear stage.

An embodiment of the first step in the process is illustrated in FIG. 18. The first actuator in this embodiment is the pair of counter-rotating wheels 360, which are actuated to move at least one of the interconnected packages/containers/sachets towards the aperture 320 in the housing. The second step in the process above is illustrated in FIG. 21. In this embodiment, the counter-rotating wheels are actuated to move the interconnected packages/containers/sachets away from the aperture 320 in the housing. The third step in the process is illustrated in FIGS. 21 and 22. In this step, the sensor arrangement 362 senses when the interconnected packages/containers/sachets are in the pre-defined starting position. The amount of actuation required to return the packages/containers/sachets to the starting position is approximated by the MCU based on the administration/delivery length, and then confirmed by the plurality of sensors. The fourth step in the process is illustrated in FIG. 22. In this step, manual access to the interconnected sachets stored within the housing may be restricted further by returning the lockout clamp 322 to the closed position. The lockout clamp 322 may be actuated by the second actuator, e.g., the solenoid.

As also illustrated in FIGS. 18 to 22, the apparatus, e.g., 300, may be part of another process that includes one or more of the following:
a. activating the actuator arrangement (e.g., the second actuator) to unlock or release a housing when the at least one sensor detects the pre-defined identifier of a pre-selected person;
b. powering the actuator arrangement (e.g., the first actuator) to move at least one of a plurality of sachets stored within the housing towards an position accessible by the person (thus moving the containers/packages from inside the housing);
c. restricting the person's access to the plurality of sachets except for the at least one sachet;
d. allowing tearing/removing, by the person, of the at least one container/package;
e. activating the actuator arrangement (e.g., the second actuator) to unlock or release the housing when the sensor detects a pre-defined identifier of the pre-selected person;
f. powering the actuator arrangement (e.g., the first actuator) to move the plurality of containers/packages away from the position accessible by the person;
g. sensing, with the sensor arrangement 362, when the plurality of containers/packages are in a pre-defined starting position; and
h. in response to the sensing of the containers/packages being in the pre-defined starting position, locking the housing to restrict the person's access to the plurality of sachets except for the at least one sachet.

As illustrated in FIG. 18, the housing 302 may be unlocked or released by actuating the second actuator to move the lockout clamp 322 to the open position. FIGS. 18 to 20 show the sachets in a position that is accessible by the person. That is, the interconnected sachets extend beyond/outside the housing 302 of the apparatus 300. As illustrated in FIGS. 19 and 20, once the sachets are in the accessible position, access to the interior of the apparatus 300 is restricted by returning the lockout clamp to the closed position. This does not prevent access to the sachet extending beyond/outside the housing 302. As illustrated in FIG. 20, the person can remove the sachet, for example, by tearing it to disconnect/remove it from the strip of interconnected sachets. Once the person has removed the sachet, they can activate the actuator arrangement (e.g., the second actuator, and in embodiments the first actuator) using the fingerprint scanner 304 to unlock or release the housing 302. As illustrated in FIG. 21, the counter-rotating wheels 360 are actuated to move the outermost interconnected sachet away from the aperture 320, and towards the interior of the apparatus 300. Once inside the housing 302, the sachets are not accessible by the person. As illustrated in FIGS. 21 and 22, the sensor arrangement 362 senses when the outermost interconnected sachet is in the pre-defined starting position. As illustrated in FIG. 22, the lockout clamp 322 may be actuated to return to the closed position in order to restrict the person's access to the plurality of sachets.

As shown in FIG. 32, the at least one sensor may include an RFID reader 3202 powered by the power source and connected to the MCU 902 to send sensed patterns/signals to the MCU 902 to indicate the data representing the tag that includes the pre-defined identifier (ID). The RFID reader may include a contactless reader/writer from NXP Semiconductors, e.g. MFRC522.

### Applications

In the field of medical sachet administration/delivery devices, or DAA Sachet Administration/Delivery Machines, the apparatus can be safe and efficient to operate because the sensor (biometric sensor and/or RFID sensor) is integrated with the activator for the administration/delivery only to the pre-selected patient, and/or because the pills/medicaments are only accessible as defined in the preselected schedule that is automatically generated from the patent medication record.

### Interpretation

Many modifications will be apparent to those skilled in the art without departing from the scope of the present invention.

The presence of “/” in a FIG. or text herein is understood to mean “and/or” unless otherwise indicated, i.e., “A/B” is understood to mean “A, or B, or both A and B”. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The terms “substantially” and “essentially all” can indicate a percentage greater than or equal to 90%, for instance, 92.5%, 95%, 97.5%, 99%, or 100%.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1.-23. (canceled)

24. An apparatus including:
a housing for containing/protecting/storing a plurality of interconnected sachets containing pills/medicaments;
an actuator arrangement mounted within the housing configured to administer/deliver the sachets containing pills/medicaments;
at least one microcontroller unit (MCU) configured to control the actuator arrangement; and
a sensor arrangement, disposed within the housing, configured to detect an edge of a leading one of the sachets and communicate with the MCU to stop the actuator arrangement when the sachets are in a starting position defined by a location of the leading edge relative to the actuator arrangement or the housing.

25. The apparatus of claim 24, wherein the sensor arrangement includes a plurality of non-contact sensors.

26. The apparatus of claim 24, wherein the actuator arrangement includes a pair of counter-rotating wheels and a motor coupled to at least one of the wheels, wherein the counter-rotating wheels in the pair are biased towards one another in order to grip the interconnected sachets therebetween.

27. The apparatus of claim 26, wherein one wheel in the pair of wheels includes gripping material to grip the interconnected sachets.

28. The apparatus of claim 26, wherein one wheel in the pair of wheels includes deformable material that deforms around the pills/medicaments in the interconnected sachets.

29. The apparatus of claim 26, wherein the wheels are configured to distribute pills/medicaments in the interconnected sachets when the pills/medicaments are mutually stacked in the interconnected sachets.

30. The apparatus of claim 24, wherein the MCU is configured to control the actuator arrangement based on a preselected administration/delivery length stored in or accessed by the MCU.

31. The apparatus of claim 24, wherein the apparatus includes an aperture through which the sachets can be administered/delivered by the actuator arrangement.

32. The apparatus of claim 31, including two pairs of counter-rotating wheels to direct the interconnected sachets straight towards the aperture.

33. The apparatus of claim 31, wherein the apparatus includes a lockout clamp movable between an open position, which opens the aperture, and a closed position, which closes the aperture to block the interconnected sachets moving through the aperture.

34. A process including:
powering an actuator arrangement to move at least one of a plurality of interconnected sachets stored within a housing towards an aperture in the housing, and/or powering an actuator arrangement to move the plurality of interconnected sachets away from the aperture in the housing;
sensing, with a plurality of sensors, when the plurality of interconnected sachets is in a pre-defined starting position; and
restricting manual access to the interconnected sachets stored within the housing.

35. The process of claim 34 further including activating the actuator arrangement to unlock or release at least one of the interconnected sachets when a sensor detects a pre-defined identifier of a pre-selected person.

36. The process of claim 34, wherein the actuator arrangement includes at least one wheel.

37. A process including:
activating an actuator arrangement to unlock or release a housing when a sensor detects a pre-defined identifier of a pre-selected person;
powering the actuator arrangement to move at least one of a plurality of sachets stored within the housing towards an position accessible by the person;
restricting the person's access to the plurality of sachets except for the at least one sachet;
allowing tearing/removing, by the person, of at least one container/package;
powering the actuator arrangement to move a plurality of containers/packages away from the position accessible by the person;
sensing, with a plurality of sensors, when the plurality of containers/packages are in a pre-defined starting position; and
in response to the sensing of the containers/packages being in the pre-defined starting position, locking the housing to restrict the person's access to the plurality of sachets except for the at least one sachet.

38. The apparatus of claim 25, wherein the actuator arrangement includes at least one pair of counter-rotating wheels and a motor coupled to at least one of the wheels, wherein the counter-rotating wheels in the pair are biased towards one another in order to grip the interconnected sachets therebetween.

39. The apparatus of claim 25, wherein the MCU is configured to control the actuator arrangement based on a preselected administration/delivery length stored in or accessed by the MCU.

40. The apparatus of claim 39, wherein the apparatus includes an aperture through which the sachets can be administered/delivered by the actuator arrangement.

41. The apparatus of claim 40, wherein the apparatus includes a lockout clamp movable between an open position, which opens the aperture, and a closed position, which closes the aperture to block the interconnected sachets moving through the aperture.

42. The process of claim 35, wherein the actuator arrangement includes at least one wheel.
